(19) United States
(12) Patent Application Publication
(10) Pub. No.: US 2010/0280382 A1
(43) Pub. Date: Nov. 4, 2010

(54) BONE FRACTURE DETECTION

(76) Inventors: Menachem Margaliot, Bnei-Brak (IL); Ronen Hareuveny, Rehovot (IL); Daniel Moran, Glil-Yam (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/811,769

(22) PCT Filed: Jan. 4, 2009
(86) PCT No.: PCT/IL09/00013

§ 371 Date: Jul. 6, 2010

Related U.S. Application Data
(60) Provisional application No. 61/006,259, filed on Jan. 3, 2008.

(51) Int. Cl. A61B 8/00; A61B 5/05

(52) U.S. Cl. 600/447; 600/407

(57) ABSTRACT

Apparatus for detecting a fracture in a bone, the apparatus comprising: an electric field generator, configured to generate an electric field in proximity of the bone, at a predefined frequency range, the generated electric field being substantially parallel to longitudinal axis of the bone; an electric field measurer, associated with the electric field generator, and configured to measure intensity values of the generated electric field over the predefined frequency range, and an analyzer, associated with the electric field measurer, and configured to analyze the intensity values measured over the predefined frequency range, thereby to detect the fracture in the bone.

### TECHNICAL FIELD

The present invention relates to bone condition detection, and, more particularly, but not exclusively to detection of fractures in bones.

Bone fractures are a common result of falling and similar accidents. The detection thereof is usually performed by traditional diagnostic X-ray devices, as well as by computed tomography (CT) X-ray scanning devices.

Hundreds of thousands of X-ray evaluations of injured bones are conducted each year in hospitals and clinics for the purpose of determining if a bone has been broken in an injury. The vast majority of these evaluations reveal normal bone, and the injury in such cases is labeled as a soft-tissue, usually trivial injury. In such cases, the X-ray evaluation is unnecessary.

However, some bone fractures, especially those known as overuse fractures, which are rather common among athletes and soldiers undergoing sudden increase in their physical activity, escape detection by the traditional X-ray devices. Usually, traditional X-ray devices fail to detect the bone fractions because of the fraction's size being smaller than the spatial resolution of the traditional X-ray device used.

Currently, detection and diagnosis of over-use fractures may be carried out by radio-isotope uptake scan. Radio-isotope uptake scan involves the administration of a dose of a bone-seeking radionuclide tracer into the body of a patient. While the fracture heals, the metabolic rate at the fracture site increases. Consequently, the radio-isotope tends to accumulates in the region of the healing fracture.

The radio-isotope uptake scan, although believed reliable, involves internal exposure of the patient to a significant dose of ionizing radiation.

Further, radio-isotope uptake scan involves the use of a very elaborate, expensive and not portable detection device, such as a Gamma Camera. A Gamma Camera is is a device used in nuclear medical imaging also known as nuclear medicine, to view and analyse images of the human body of the distribution of medically injected, inhaled, or ingested gamma ray emitting radio-nuclides

Ultrasonic methods are also used in bone density and strength measurement.

For example, U.S. Pat. No. 3,847,141, to Hoop, filed on Aug. 8, 1973, entitled “Ultrasonic Bone Densitometer”, discloses an ultrasonic method for assessment of calcium content in an examined bone. The method described by Hoop is based on preferential frequency transmission. The preferential frequency transmission is claimed to be calcium dependent.

U.S. Pat. No. 4,361,154 to Pratt, entitled “Method for establishing, in vivo, bone strength”, filed on Jun. 15, 1979, describes using ultrasound waves at two frequencies, transmitted along an examined bone. Pratt determines the bone integrity from the sound velocity of propagation via a given length of limb.

However, the use of ultrasonic methods to detect bone fractures in general, and small bone fractures in particular has been limited by strong interference by soft tissue, which overlays the bone.

Electric impedance has also been proposed for examination of bone conditions.

For example, U.S. Pat. No. 5,782,763, to Bianco et al., filed on Jul. 1, 1997, entitled “Electromagnetic bone-assessment apparatus and method”, describes the application of electric fields in the 0-200 Mhz frequency range to a bone, perpendicularly to the bone's long axis.

According to the method described by Bianco, the voltage/current values obtained are analyzed (using Fourier Transformations), to yield bone structure information. Then, the bone structure information is used to diagnose Osteoporosis.

A very similar approach is presented by Bianco et al. in U.S. Pat. No. 6,213,934, filed on Apr. 30, 1998, where a 0-200 Mhz frequency range magnetic field is used rather than an electric field.

However, both Bianco methods mentioned above are extremely sensitive to effects of overlaying soft tissue.

The overlaying soft tissue has a higher than bone conductivity. Consequently, the overlaying soft tissue has a detrimental effect on the quality of measurements, and introduces distortion and uncertainty into results obtained using the measurements described by Bianco.

Further, the calibration process of the aforementioned methods is based on a comparison of obtained bone (and more specifically, limb) results, to results previously obtained on a reference fixed specimen. The calibration process introduces variations due to geometrical and structural differences between same bones (and more specifically, limbs) in different persons.

There is thus a widely recognized need for, and it would be highly advantageous to have, a system and method devoid of the above limitations.

### SUMMARY OF INVENTION

According to one aspect of the present invention there is provided an apparatus for detecting a fracture in a bone, the apparatus comprising: an electric field generator, configured to generate an electric field in proximity of the bone, at a predefined frequency range, the generated electric field being substantially parallel to longitudinal axis of the bone; an electric field measurer, associated with the electric field generator, and configured to measure intensity values of the generated electric field over the predefined frequency range, and an analyzer, associated with the electric field measurer, and configured to analyze the intensity values measured over the predefined frequency range, thereby to detect the fracture in the bone.

According to a second aspect of the present invention there is provided a method for detecting a fracture in a bone, the method comprising: generating an electric field in proximity of the bone, at a predefined frequency range, the generated electric field being substantially parallel to longitudinal axis of the bone; measuring intensity values of the generated electric field over the predefined frequency range; and analyzing the intensity values measured over the predefined frequency range, thereby to detect the fracture in the bone.

According to a third aspect of the present invention there is provided a method for detecting a fracture in a bone, the method comprising: obtaining reference intensity values; generating an electric field in proximity of the bone, at a predefined frequency range, the generated electric field being substantially parallel to longitudinal axis of the bone; measuring intensity values of the generated electric field over the predefined frequency range; and analyzing the intensity values measured over the predefined frequency range using the obtained reference intensity values, thereby to detect fracture in the bone.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

Moreover, according to actual instrumentation and equipment of preferred embodiments of the method and system of the present invention, several selected steps could be implemented by hardware or by software on any operating system of any firmware or a combination thereof For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF DRAWINGS

The invention is herein described, by way of example only, with reference to the accompanying drawings.

With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention.

The description taken with the drawings making apparent to those skilled in the art how the several forms of the invention may be embodied in practice.

In the drawings:

FIG. 1 is a simplified block diagram illustrating a bone fracture.

FIG. 2 is a simplified block diagram illustrating a first apparatus for detecting a fracture in a bone, according to a preferred embodiment of the present invention.

FIG. 3 is a simplified block diagram illustrating a second apparatus for detecting a fracture in a bone, according to a preferred embodiment of the present invention.

FIG. 4 is a flowchart illustrating a first method for detecting a fracture in a bone, according to a preferred embodiment of the present invention.

FIG. 5 is a flowchart illustrating a second method for detecting a fracture in a bone, according to a preferred embodiment of the present invention.

FIG. 6a illustrates an exemplary in vitro model of a healthy bone, according to a preferred embodiment of the present invention.

FIG. 6b illustrates an exemplary in vitro model of a fractured bone, according to a preferred embodiment of the present invention.

FIG. 7 illustrates an exemplary in vitro model of a bone, installed with electrodes, according to a preferred embodiment of the present invention.

FIG. 8 illustrates a first exemplary spectrum obtained from an exemplary in vitro model of a healthy bone, according to a preferred embodiment of the present invention.

FIG. 9 illustrates a first exemplary spectrum obtained from an exemplary in vitro model of a fractured bone, according to a preferred embodiment of the present invention.

FIG. 10 illustrates an exemplary calculated spectrum ratio chart, according to a preferred embodiment of the present invention.

### DESCRIPTION OF EMBODIMENTS

The present embodiments comprise an apparatus and a method for detecting a fracture in a bone.

A method according to a preferred embodiment of the present invention may be used to detect fracture in a bone, such as a human limb.

According to a preferred embodiment, an electric field is generated at a predefined frequency range. For example, a frequency range in the 200 MHz-10 GHz range is optimized for the length of a human foot or arm, whereas a frequency range of 100 MHz-20 GHz is useful for humans as well as some animals smaller or larger than man.

The field is generated along the examined bone (say, the human limb). That is to say, the electric field is generated substantially parallel to longitudinal axis of the bone.

The generated electric field's intensity values as a function of frequency are measured over the predefined frequency range.

Finally, the frequency dependence of the generated electric field's intensity is analyzed, and used to detect fracture in the bone. For example, there may identified one or more frequencies at which the intensity of the electric field is sharply attenuated, as described in further detail hereinbelow.

In a preferred embodiment, the electric field is generated and measured at the same frequency range, on both legs of a same person. Then, the frequency dependencies (say energy absorption spectrums, as known in the art) of the generated field of each of the two legs are compared.

A difference between the two frequency dependencies may be indicative of fracture, as described in further detail hereinbelow.

Preferably, if it is suspected that both of the legs have fractures, the frequency dependencies of one or two of the legs may be compared to frequency dependency of a leg of a healthy person, or an averaged frequency dependency calculated from several healthy persons.

The principles and operation of an apparatus and method according to the present invention may be better understood with reference to the drawings and accompanying description.

Before explaining at least one embodiment of the invention in detail, it is to be understood that the invention is not limited in its application to the details of construction and the arrangement of the components set forth in the following description or illustrated in the drawings.

The invention is capable of other embodiments or of being practiced or carried out in various ways. Also, it is to be understood that the phraseology and terminology employed herein is for the purpose of description and should not be regarded as limiting.

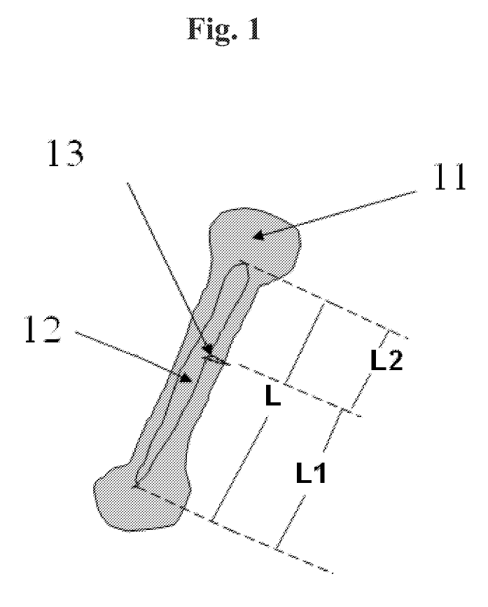

Reference is now made to FIG. 1, which is a simplified block diagram illustrating a bone fracture.

A bone, say one of the long bones of the leg, has an external tubular, hard, and relatively dry part 11.

The bone's inner part, contained within the external tubular part 11, consists of bone marrow 12. The bone marrow 12 is a relatively wet tissue, with high water content.

The bone is further enclosed by soft tissue (muscle, skin, blood vessels, etc.) of larger size (length) than the bone.

The sharp difference in water content between the external 11 and inner parts 12 of the bone, results in differences in the electric properties of the bone parts, say in conductivity of the parts of the bone.

Due to the bone marrow's relatively high water content, the bone marrow 12 may be considered an electrical conductor of length L enclosed in an insulator—the external part of the bone 11. For convenience of presentation, the long bones of the leg, due to their dominant longitudinal axis, may be considered a one-dimensional electrical conductor.

When a time-varying electric field is applied to a conductor of final length, the current induced in the conductor has a significant frequency dependency, peaking at a specific resonant frequency.

The resonant frequency is given by the equation 1:

f=C/2L (Equation 1)

Where f—is the resonant frequency, C—is the propagation velocity of electric current in the conductor, and L—is the length of the conductor.

When such a conductor is not fully isolated, but rather has an electric contact at some point along its longitudinal axis, the conductor is short circuited on the point.

That is to say, the electric contact divides the conductor into two parts, L1 and L2, where L=L1+L2. Consequently, in addition to the aforementioned main resonant frequency f (resonant)=C/2L, two new resonant frequencies appear:

f₁=C/2L1 and f₂=C/2L2.

The appearance of the additional resonant frequencies is indicative of the existence of a localized conduction path in the bone. The localized conduction path electrically connects the bone marrow 12 and the soft tissue which surrounds the bone.

Optionally, the localized conduction path is created by a fracture 13, such as a tension fracture present in the bone. The fracture 13 creates the localized conduction path, which electrically connects the bone marrow 12 and the soft tissue which surrounds the bone.

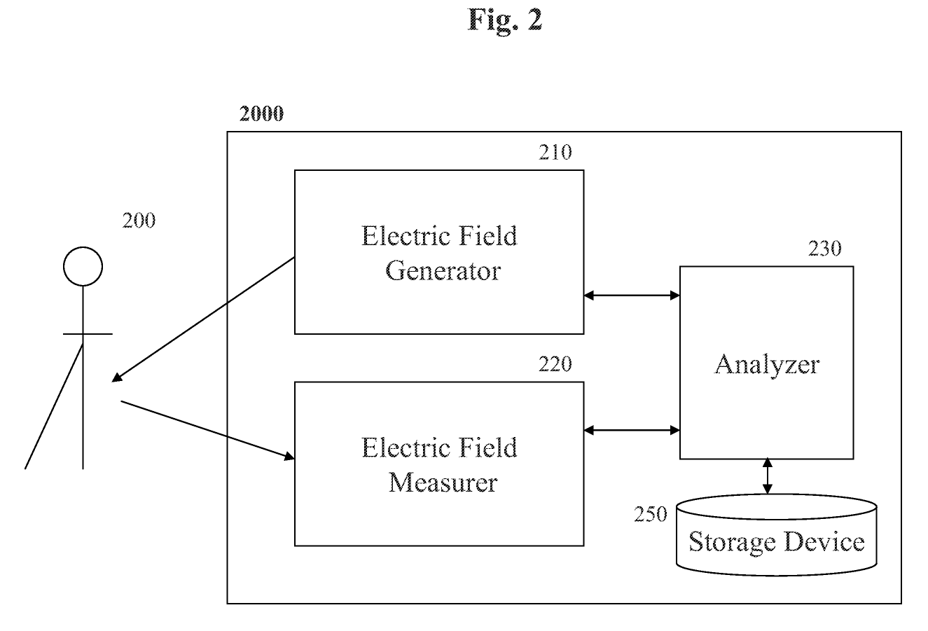

Reference is now made to FIG. 2 which is a simplified block diagram illustrating a first apparatus for detecting a fracture in a bone, according to a preferred embodiment of the present invention.

Apparatus 2000 includes an electric field generator 210.

The electric field generator 210 generates an electric field in proximity of a long bone, such as a person's limb 200, at a predefined frequency range. The generated electric field is substantially parallel to longitudinal axis of the bone.

Optionally, the electric field generator 210 is a Radio-Frequency (RF) signal generator, as known in the art.

Optionally, apparatus 2000 also includes two or more electrodes.

The two electrodes are connected to the electric field generator 210, as described in further detail hereinbelow. The electrodes may be deployed along the longitudinal axis of the bone.

Apparatus 2000 further includes an electric field measurer 220. The electric field measurer 220 measures intensity values of the generated electric field over the predefined frequency range.

Optionally, the apparatus 2000 further includes one or more electrode(s) connected to the electric field measurer 220. The electrode(s) connected to the electric field measurer 220 may be deployed along the longitudinal axis of the bone.

Preferably, the electrode(s) connected to the electric field may be moved along the longitudinal axis of the bone, as described in further detail hereinbelow.

For example, the electrode(s) connected to the electric field measurer may be moved along the bone, into a position where the attenuation of the electric field is maximal. The position where the attenuation is maximal is indicative of the exact position of the fracture in the bone, as described in further detail hereinbelow.

Apparatus 2000 further includes an analyzer 230, connected to the electric field generator 210 and the electric field measurer 220.

The analyzer 230 analyzes the intensity values measured over the predefined frequency range, thereby to detect fracture in the bone.

For example, the analyzer 230 may identify frequencies at which the electric field is sharply attenuated. The frequencies at which the electric field is sharply attenuated may be indicative of a fracture in the bone, as described in further detail hereinbelow.

Preferably, the frequency range is selected so as to generate a wavelength optimized for the length of the bone.

For example, a frequency range in the 200 MHz-10 GHz range is optimized for the length of a human foot or arm, whereas a frequency range of 100 MHz-20 GHz is useful for humans as well as for some animals smaller or larger than man.

Optionally, the analyzer 230 controls the electric field generator 210, the electric field measurer 220, or both, as described in further detail hereinbelow.

Preferably, the apparatus 2000 also includes a storage device 250, connected to the analyzer 230.

The analyzer 230 may store the intensity values measured over the predefined frequency range on the storage device 250.

Optionally, the analyzer 230 compares the intensity values measured over the predefined frequency range with reference intensity values stored on the storage device 250 in advance of the generation of the electric field.

In one example, the bone may be a first limb (say a foot or an arm) of a person. A comparison may be carried out between the intensity values measured over the limb, and reference intensity values.

Optionally, the reference intensity values are measured over the limb's twin limb (say a foot or an arm) of the same person. The reference intensity values are stored on the storage device 250, in advance of the measurement of the intensity values over the first limb of the person.

The comparison may help detect the fracture in one of the person's limbs, as described in further detail hereinbelow.

In a second example, it is suspected that a first person has fractures in both his feet. A comparison is made between the intensity values measured over each the first person's feet and reference intensity values previously measured over a healthy person's foot (or averaged values measure over several healthy individuals).

The comparison may help detect the fracture in one of the person's feet, or both of the person's feet, as described in further detail hereinbelow.

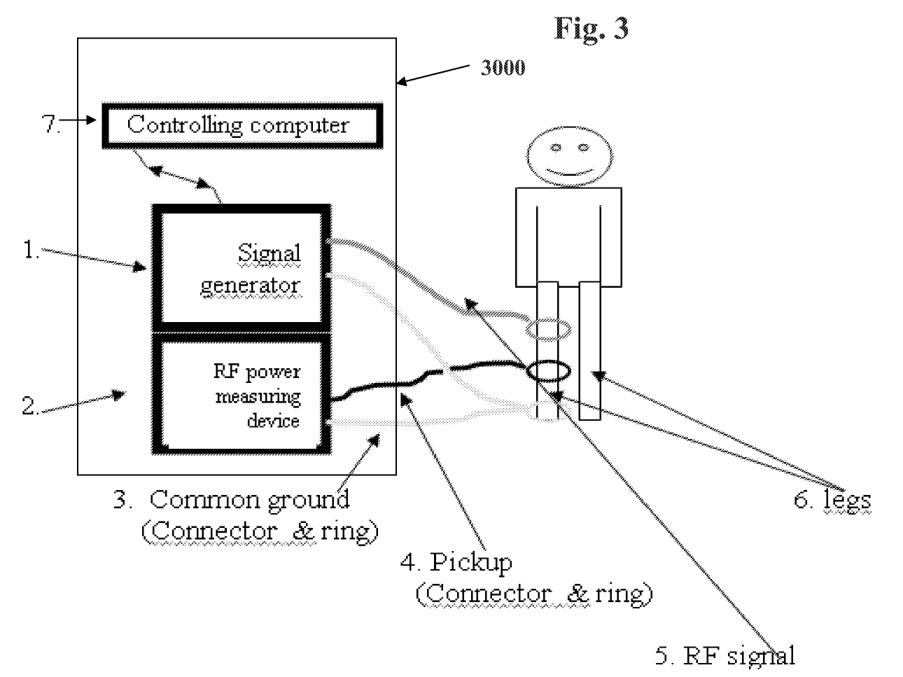

Reference is now made to FIG. 3, which is a simplified block diagram illustrating a second apparatus for detecting a fracture in a bone, according to a preferred embodiment of the present invention.

Apparatus 3000 includes an electric field generator implemented as a Radio-Frequency (RF) signal generator 1 (or an equivalent device such as an electric circuit, or a network analyzer), as known in the art.

The Radio-Frequency (RF) signal generator 1 generates an alternating electric field in proximity of a long bone, such as a parson's foot 6, at a predefined frequency range, as described in further detail hereinabove. The generated electric field is substantially parallel to longitudinal axis of the bone.

Optionally, a Radio-Frequency (RF) signal produced by the Radio-Frequency (RF) signal generator 1 is fed via an electric cable, to an electrode 5 placed in the near proximity of one edge of an examined bone 6. Consequently, there is generated the alternating electric field along the longitudinal axis of the bone, as described in further detail hereinbelow.

The electrode 5 fed with the RF signal is herein referred to as the input electrode 5.

Apparatus 3000 further includes a common ground electrode 3, as known in the art.

The common ground electrode 3 is placed in near proximity of one edge of the examined bone 6. The alternating electric field is generated between the input electrode 5 and the common ground electrode 3.

Apparatus 3000 further includes an electric field measurer implemented as a Radio-Frequency (RF) power measurement device 2, such as a RF power meter, a spectrum analyzer, a dedicated circuit incorporated in the system, a network analyzer, or any combination thereof, as known in the art.

The RF power measurement device 2 is electrically connected to an electrode 4 placed in the near proximity of the examined bone, at approximately the middle of the bone 6. The electrode 4 is herein referred to as the pick-up electrode 4.

The pick-up electrode 4 senses the electric field generated by the Radio-Frequency (RF) signal generator 1. Optionally, the pick-up electrode is deployed in a mid-point along the bone. Preferably, the pick-up electrode is movable along the bone, as described in further detail hereinbelow.

Optionally, the pick-up electrode 4 is a coil wounded around the bone, as known in the art. The coil wounded around the bone senses electric currents induced in the bone upon the generation of the electric field.

The common ground electrode 3 is electrically connected to the common ground of both the Radio-Frequency (RF) signal generator 1 and the Radio-Frequency (RF) power measurement device 2.

Apparatus 3000 further includes an analyzer implemented as a controlling computer 7.

The controlling computer 7 is electrically connected to both the Radio-Frequency (RF) signal generator 1 and the Radio-Frequency (RF) power measurement device 2.

The controlling computer 7 initiates a measurement sequence by commanding the Radio-Frequency (RF) signal generator 1 to generate Radio-Frequency (RF) signals at several selected frequencies of a predefined range of frequencies, as described in further detail hereinabove.

The controlling computer 7 receives the intensity values picked up by the pickup electrode 4 and measured by the Radio-Frequency (RF) power measurement device 2, at each one of the selected frequencies of the predefined frequency range.

The controlling computer 7 analyzes the measured intensity values, and stores results of the analysis in a form of measured intensity versus frequency, say as an absorption spectrum, as described in further detail hereinbelow.

The controlling computer 7 further initiates a second measurement sequence, for the second foot of the examined person. Intensity values measured during the second measurement sequence are also stored in a form of measured intensity versus frequency, such as an absorption spectrum.

Then, the controlling computer 7 compares the two absorption spectrums.

The comparison may show a significant difference between the two spectrums. For example, the difference may be an additional intensity value attenuation peak present in an absorption spectrum of one of the feet (i.e. a minimal intensity value), as described in further detail hereinbelow.

The difference serves as an indication of a fracture present in one of the feet.

Preferably, the pick-up electrode 4 is movable between different positions along the foot, say by sliding the pick-up electrodes between the different positions, as described in further detail hereinbelow.

Usually, a position of the movable pick-up electrode at which the difference between the two spectrums is most significant is a position in closest vicinity of the fracture.

Preferably, the Radio-Frequency (RF) signal generator 1 includes the input electrode 5 deployed just beneath a person's knee, and the common ground electrode 3 deployed just above the person's ankle.

Optionally, the electrodes 3, 5 are made of copper foil. The foil is one centimeters wide, and one millimeter thick, and has a diameter of fifteen centimeters.

Alternatively, the electrodes 3, 5 may be made of other conductive materials, be the materials metallic or other, and have different diameters, thicknesses, or widths.

The electrodes 3, 5 may be positioned in proximity of the person's leg.

Optionally, the electrodes 3, 5 are ring electrodes, which encircle the leg, in close proximity to the person's leg, without being in electric contact with the tissue of the leg.

Optionally, the diameter of each of the ring electrodes 3, 5 is only a little (say a few millimeters) larger than the diameter of the leg.

Preferably, for convenience application, the ring electrodes 3, 5 may be wound over a non-conductive tube into which the leg is inserted. The tube is made of a nonconductive material, such as plastic, etc.

Optionally, the Radio-Frequency (RF) signal generator 1 further includes a Radio-Frequency (RF) signal source, such as a Radio-Frequency (RF) Oscillator, or any other RF signal source, as known in the art.

Optionally, the Radio-Frequency (RF) signal source is capable of producing an electric signal at the predefined frequency range (say 200 MHz-10 GHz, as described in further detail hereinabove).

Optionally, the Radio-Frequency (RF) signal source provides low power alternating electric signal in the predefined frequency range (say 0.2-10 GHz).

The common ground electrode 3 is electrically connected to the common ground of both the Radio-Frequency (RF) signal generator 1 and the Radio-Frequency (RF) power measurement device 2. That to say that the common ground of the Radio-Frequency (RF) signal generator 1 is used as the common ground the whole apparatus 4000.

The input electrode 5 is electrically connected to the RF signal source, while the ground electrode 3 is electrically connected to a common ground of the system.

Consequently, an alternating electric field is formed between the two electrodes 3,5. The electric field alternates at frequencies of the predefined frequency range, say between 200 MHz and 10 GHz, as described in further detail hereinabove.

Optionally, pick-up electrode 4 connected to the Radio-Frequency (RF) intensity measuring device 2 is wounded on the none-conductive tube described hereinabove.

Optionally, the pick-up electrode 4 is a coil wounded around the bone. The coil senses the electric currents induced in the bone upon the generation of the alternating electric field.

Optionally, the pick-up electrode 4 is a ring electrode positioned between the two ring-electrodes 3, 5 connected to the Radio-Frequency (RF) signal generator 1.

Optionally, pick-up electrode 4 the Preferably, the pick-up electrode 4 may slide along the tube the leg us inserted to. The pick-up electrode 4 is thus movable between positions along the bone.

Preferably, the Radio-Frequency (RF) intensity measuring device 2 includes a Radio-Frequency (RF) power measuring device, as known in the art.

The radio-Frequency (RF) power measurement device 2 is electrically connected to the input of the pick-up electrode 4. The radio-Frequency (RF) power measurement device 2 measures the intensity of electric field in the predefined frequencies range.

Preferably, the controlling computer 7 is electrically connected to the Radio-Frequency (RF) signal source. The controlling computer 7 controls the Radio-Frequency (RF) signal source and determines the frequency generated at each given moment.

Preferably, the controlling computer 7 is further electrically connected to the radio-Frequency (RF) power measurement device 2.

The controlling computer 7 records the intensity values of the electric field (e.g.—amplitude), as sensed by the pickup electrode 4, and measured by the RF power measurement device 2, at any specific frequency in the predefined frequency range.

Preferably, the controlling computer 7 may also direct the operator of the system to conduct another sequential measurement on a second leg of the person.

The controlling computer 7 may use the intensity values measured over the first leg as reference values, and compare the reference intensity with values measured over the second leg.

The presence of significant differences between the intensity values measured over the first leg and intensity values measured over the second leg is indicative of an existence of a fracture in one of the person's legs.

Preferably, the controlling computer 7 further finds out at which position of the pick-up electrode (which slides along the tube) the differences are maximal. The position where the differences are maximal is indicative of the location of the fracture in the apparently injured leg.

In an exemplary measurement procedure, the tube is placed over one leg of the examined person.

The RF source generates a low-power RF signal at a frequency within the above-mentioned predefined frequency range, preferably in a sweeping manner (that is to say that the RF source scans the frequency range).

Using the input electrode 5, there is generated an electric field along the longitudinal axis of the bone, as described in further detail hereinabove.

The generated electric field's intensity value at each specific frequency as sensed by the pick-up electrode 4 and measure by the RF power measurement device 2 is fed to the controlling device 7. The controlling computer 7 records the intensity values as a function of the frequency, say as an absorption spectrum.

A similar measurement procedure in repeated over the other leg of the same examined person, and the controlling computer 7 records the intensity values measured over the second leg, as a second absorption spectrum.

The controlling computer 7 compares the two absorption spectrums.

Typically, the outcome of the comparison reveals the presence of differences between absorption peaks in the absorption spectrum of the healthy leg, and absorption peaks in the absorption spectrum of the spectrum of the leg suspected as injured.

The presence of (typically) two or more different peaks is indicative of a fracture present in the leg, as described in further detail hereinbelow.

Preferably, the pick-up electrode 4 is moved between different positions along the tube.

Preferably, the measurements are made at different positions (i.e. heights) of the pick-up electrode 4 along each of the two legs. Then, there is carried out the above described comparison, for each pair of the pick-up electrode's positions, at the same height of each of the two legs. The pair of positions where the differences between the two legs are most significant are indicative of the location (i.e. height) of the fracture in the bone.

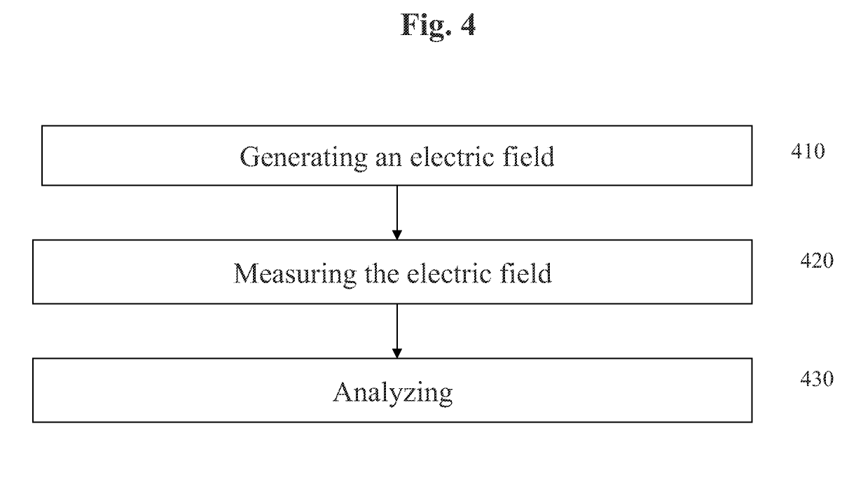

Reference is now made to FIG. 4, which is a flowchart illustrating a first method for detecting a fracture in a bone, according to a preferred embodiment of the present invention.

In a method according to a preferred embodiment of the present invention, there is generated 410 an electric field in proximity of a long bone (such as a parson's limb).

The electric field is generated at a predefined frequency range, say using the Radio-Frequency (RF) signal generator 1, as described in further detail hereinabove. The generated electric field is substantially parallel to longitudinal axis of the bone.

The intensity values of the generated electric field are measured 420 over the predefined frequency range, say using the RF intensity measuring device 2, as described in further detail hereinabove.

The intensity values measured over the predefined frequency range are analyzed 430, thereby to detect fracture in the bone, say by identifying frequencies at which the electric field is sharply attenuated, as described in further detail hereinbelow.

Preferably, the frequency range is selected so as to generate a wavelength optimized for the length of the bone.

For example, a frequency range in the 200 MHz-10 GHz range is optimized for the length of a human foot or arm, whereas a frequency range of 100 MHz-20 GHz is useful for humans as well as some animals smaller or larger than man.

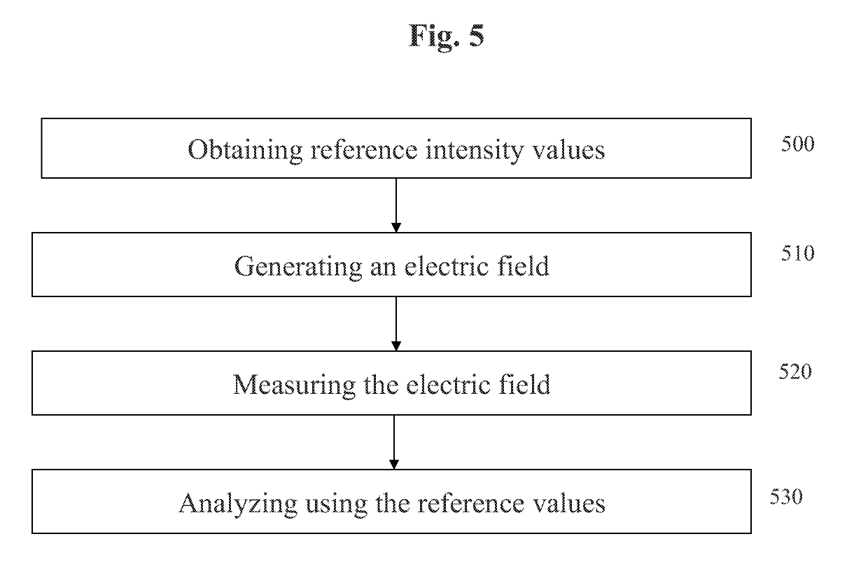

Reference is now made to FIG. 5, which is a flowchart illustrating a second method for detecting a fracture in a bone, according to a preferred embodiment of the present invention.

According to a preferred embodiment, there are obtained 500 reference intensity values, to be used for analysis by comparison of intensity values measured over the bone, as described in further detail hereinbelow.

In a first example, the bone may be a first limb (say a foot or an arm) of a person. The comparison is carried out between the intensity values measured over the limb, and reference intensity values.

The reference intensity values are measured over the limb's twin limb (say a foot or an arm) of the same person, and obtained in advance of the measurement of the intensity values over the first limb of the person.

In a second example, it is suspected that a first person has fractures in both his feet. A comparison is made between the intensity values measured over each of the first person's feet and reference intensity values measured over a healthy person's foot (or averaged values calculated using intensity values measure over several healthy individuals' feet).

Next, there is generated 510 an electric field in proximity of the bone, such as a parson's limb.

The electric field is generated at a predefined frequency range, say using the Radio-Frequency (RF) signal generator 1, as described in further detail hereinabove. The generated electric field is substantially parallel to longitudinal axis of the bone.

The intensity values of the generated electric field are measured 520 over the predefined frequency range, say using the Radio-Frequency (RF) intensity measuring device 2, as described in further detail hereinabove.

The intensity values measured over the predefined frequency range are analyzed 530 using reference values, say by comparison to the reference values previously measured over the predefined frequency range, as described in further detail hereinabove.

The comparison may help detect a fracture in one of the person's limbs, as described in further detail hereinabove.

It is expected that during the life of this patent many relevant devices and systems will be developed and the scope of the terms herein, particularly of the terms “Radio-Frequency (RF)”, “Intensity measuring device”, and “Radio-Frequency (RF) signal generator”, is intended to include all such new technologies a priori.

Additional objects, advantages, and novel features of the present invention will become apparent to one ordinary skilled in the art upon examination of the following examples, which are not intended to be limiting.

Additionally, each of the various embodiments and aspects of the present invention as delineated hereinabove and as claimed in the claims section below finds experimental support in the following example.

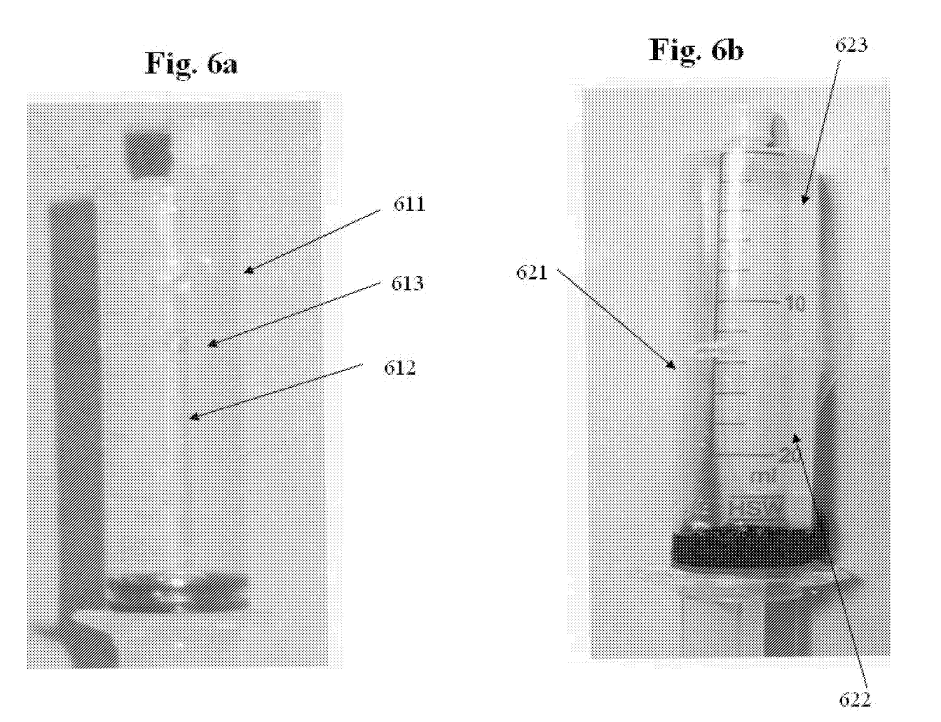

Reference is now made to FIG. 6a, illustrates an exemplary in vitro model of a healthy bone, according to a preferred embodiment of the present invention.

An exemplary in vitro model of a bone includes a first volumetric cylinder 611.

The first volumetric cylinder 611 is filled with a Ringer Solution. A Ringer Solution is an aqueous solution which contains chlorides of sodium and potassium, and calcium. The solution is isotonic to animal tissues and conductive (as it contains ions).

Inside the first volumetric cylinder 611, there is installed a thin syringe 612 made of glass.

The syringe 612 is also filled with the Ringer solution.

The solution inside the syringe 612 models the bone marrow, whereas the solution in the space 613 between the syringe and the first cylinder's 611 wall models the soft tissue which overlays the bone.

The body of the syringe 612 models the dry and nonconductive external tubular part of the bone. The bone marrow is contained within the external tubular part, as described in further detail hereinabove.

Reference is now made to FIG. 6b, which illustrates an exemplary in vitro model of a fractured bone, according to a preferred embodiment of the present invention.

An exemplary in vitro model of a fractured bone includes a second volumetric cylinder 621 filled with a Ringer solution.

Inside the second volumetric cylinder 621, there is installed a thin syringe 622 made of glass.

The syringe 622 is also filled with the Ringer solution.

The solution inside the syringe 622 models the bone marrow, whereas the solution in the space 623 between the syringe the second cylinder's 621 wall models the soft tissue which overlays the bone.

The body of the syringe 622 models the dry and nonconductive external tubular part of the bone. The bone marrow is contained within the external tubular part, as described in further detail hereinabove.

In order to model the fracture, the syringe 622 is cracked, so as to allow contact between the solution inside the syringe 622 and the solution in the space 623 between the syringe and the second cylinder's 621 wall.

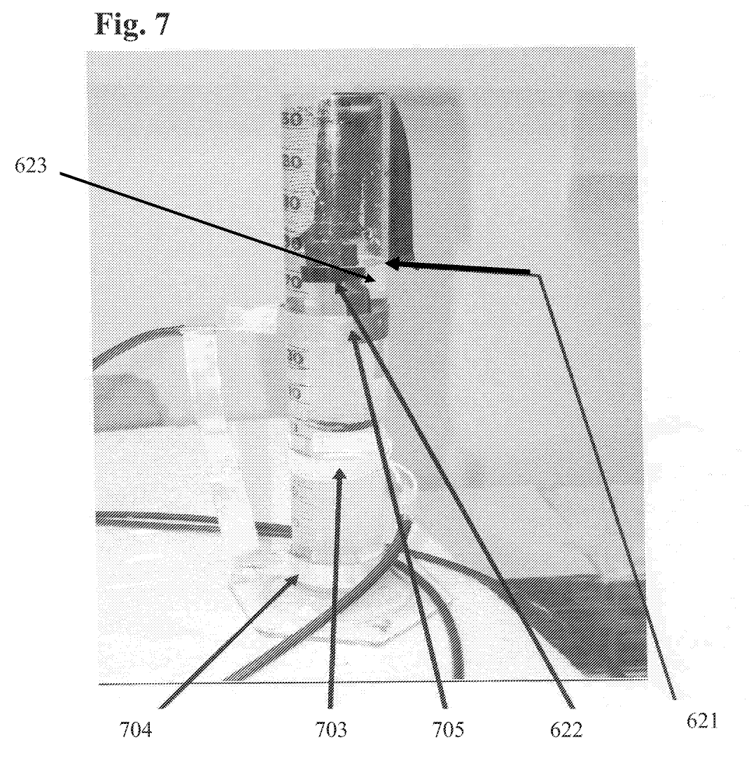

Reference is now made to FIG. 7, which illustrates an exemplary in vitro model of a bone, installed with electrodes, according to a preferred embodiment of the present invention.

On each of cylinders 611, 621, mentioned hereinabove, there are installed input 705, pick-up 703 and ground electrodes 704, as described in further detail hereinabove.

Using the electrodes 703-705, the electric field generator 210, and the electric field measurer 220, there are generated alternating electric fields along each of the cylinders 611, 621.

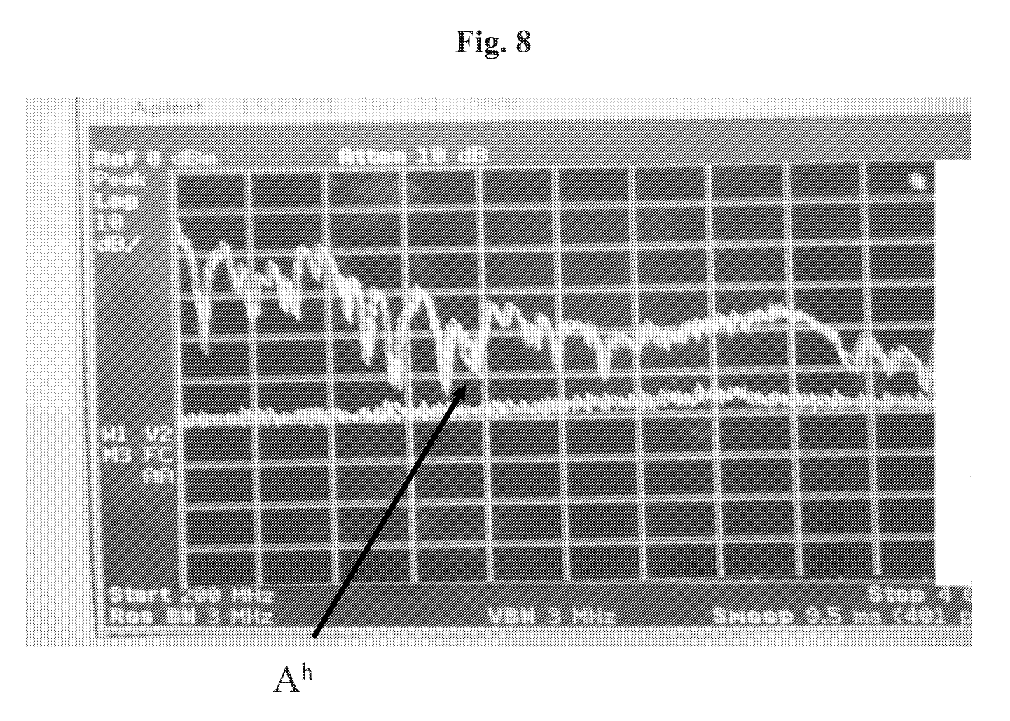

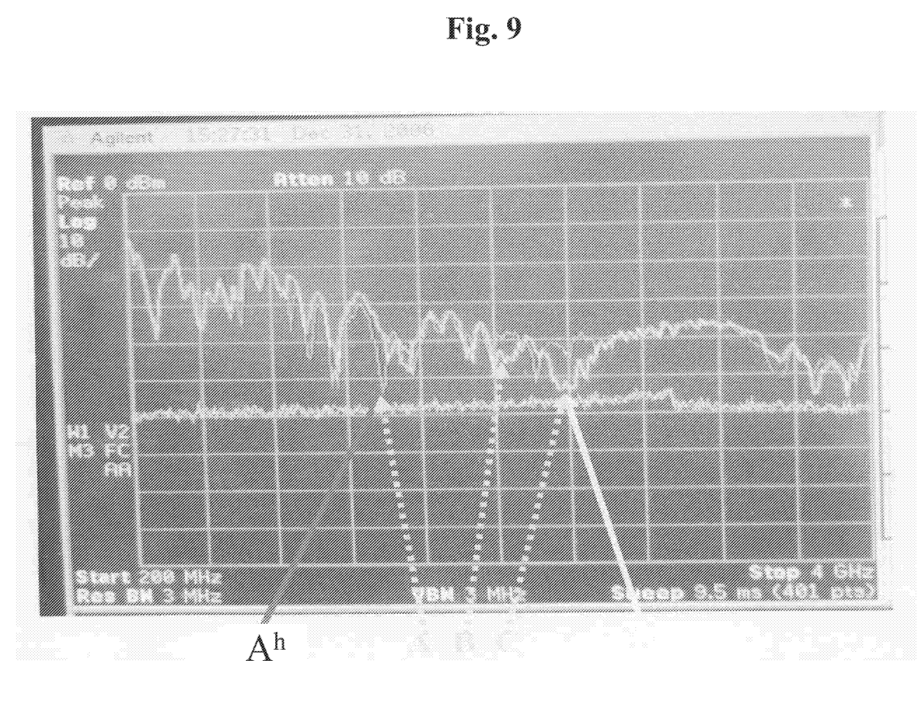

The fields' intensity values over a predefined frequency range are measured and analyzed, yielding intensity over frequency spectrums, as described in further detail hereinabove. The frequency spectrums are illustrated using FIGS. 8 and 9 hereinbelow.

Reference is made to FIG. 8, which illustrates a first exemplary spectrum obtained from an exemplary in vitro model of a healthy bone, according to a preferred embodiment of the present invention.

A measurement and analysis of the intensity values of electric field generated along the first cylinder 611, which models a healthy bone, yields a spectrum as illustrated in FIG. 8.

The spectrum of FIG. 8 is presented in a logarithmic presentation. The spectrum of FIG. 8 has a significant absorption peak Ah.

Reference is made to FIG. 9, which illustrates a second exemplary spectrum obtained from an exemplary in vitro model of a fractured bone, according to a preferred embodiment of the present invention.

A measurement and analysis of the intensity values of electric field generated along the second cylinder 621, which models a fractured bone yields a spectrum as illustrated in FIG. 9. The spectrum of FIG. 9 is presented in a logarithmic presentation.

The most significant difference between the spectrum illustrated in FIG. 8, and the spectrum illustrated in FIG. 9 is the appearance of two new absorption peaks B,C, and the significant weakened peak A replacing the absorption peak Ah obtained from the healthy bone model.

The differences are the results of the short circuiting or shunting between the solution inside the syringe 622 (the solution inside the syringe models the bone marrow) and the solution in the space 623 between the syringe 622 and the second volumetric cylinder 621 (the solution in the space 623 models the soft tissue overlaying the bone).

In vivo, the short-circuiting is facilitated by the fracture in bone, as described in further detail hereinabove.

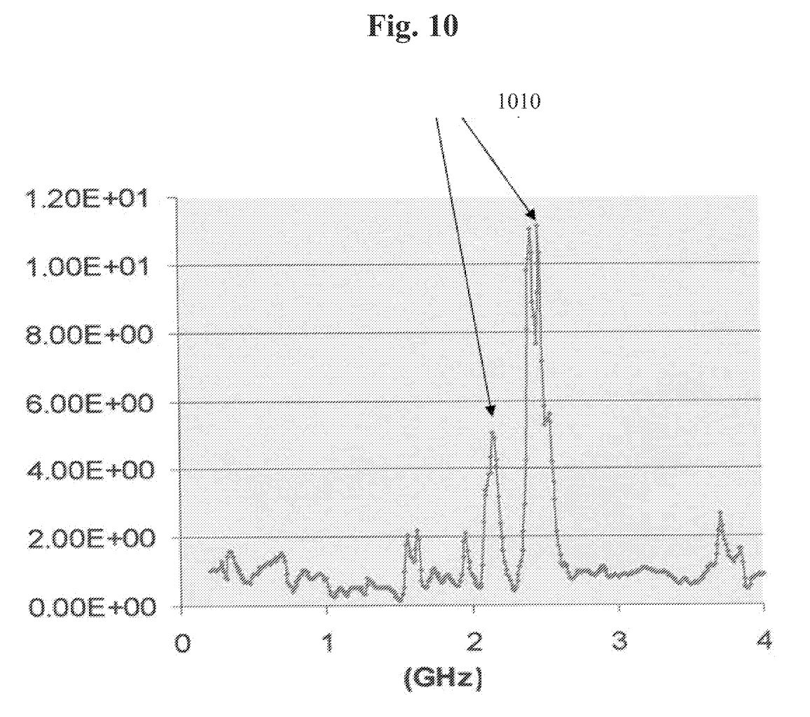

Reference is made to FIG. 10, which illustrates an exemplary calculated spectrum ratio chart, according to a preferred embodiment of the present invention.

An exemplary calculated spectrum ratio chart 1000 represents the intensity ratios of the spectrum obtained from the model of a healthy bone (as illustrated hereinabove, using FIG. 8), and the spectrum obtained from the model of a fractured bone (as illustrated hereinabove, using FIG. 9).

The exemplary spectrum ratio chart 1000 clearly shows the differences between the two spectrums, in a form of clear peaks 1010, which correspond to the three absorption peaks, as described in further detail hereinabove.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. Apparatus for detecting a fracture in a bone, the apparatus comprising:
an electric field generator, configured to generate an electric field in proximity of the bone, at a predefined frequency range, said generated electric field being substantially parallel to longitudinal axis of the bone;
an electric field measurer, associated with said electric field generator, and configured to measure intensity values of said generated electric field over said predefined frequency range, and
an analyzer, associated with said electric field measurer, and configured to analyze said intensity values measured over said predefined frequency range, thereby to detect the fracture in the bone.

2. The apparatus of claim 1, wherein said frequency range is between 100 MHz and 20 GHz.

3. The apparatus of claim 1, wherein said frequency range is between 200 MHz and 10 GHz.

4. The apparatus of claim 1, wherein said electric field generator is a Radio-Frequency (RF) signal generator.

5. The apparatus of claim 1, wherein said apparatus further comprises at least two electrodes, deployable along said longitudinal axis of the bone and associated with said electric field generator.

6. The apparatus of claim 5, wherein said two electrodes are ring electrodes.

7. The apparatus of claim 1, wherein said apparatus further comprises at least one electrode deployable along said longitudinal axis of the bone and associated with said electric field measurer.

8. The apparatus of claim 7, wherein said electrode associated with said electric field measurer is movable along said longitudinal axis of the bone.

9. The apparatus of claim 7, wherein said electrode associated with said electric field measurer is a ring electrode.

10. The apparatus of claim 1, wherein said analyzer is further configured to control said electric field generator.

11. The apparatus of claim 1, wherein said analyzer is further configured to control said electric field measurer.

12. The apparatus of claim 1, further comprising a storage device, associated with said analyzer, wherein said analyzer is further configured to store said intensity values measured over said predefined frequency range on said storage device.

13. The apparatus of claim 1, further comprising a storage device, associated with said analyzer, wherein said analyzer is further configured to compare said intensity values measured over said predefined frequency range with reference intensity values stored on said storage device in advance of said generation of said electric field, thereby to detect fracture in the bone.

14. The apparatus of claim 13, wherein the bone is a first limb of a person, and said reference intensity values are intensity values previously obtained from a second limb of said person.

15. The apparatus of claim 13, wherein the bone is a limb of a first person, and said reference intensity values are intensity values previously obtained from a limb of a second person.

16. Method for detecting a fracture in a bone, the method comprising:
generating an electric field in proximity of the bone, at a predefined frequency range, said generated electric field being substantially parallel to longitudinal axis of the bone;
measuring intensity values of said generated electric field over said predefined frequency range; and
analyzing said intensity values measured over said predefined frequency range, thereby to detect the fracture in the bone.

17. The method of claim 16, wherein said frequency range is between 200 MHz and 10 GHz.

18. The method of claim 16, wherein said frequency range is between 100 MHz and 20 GHz.

19. The method of claim 16, further comprising storing said intensity values measured over said predefined frequency range on a storage device.

20. The method of claim 16, further comprising comparing said intensity values measured over said predefined frequency range with reference intensity values stored on a storage device in advance of said generation of said electric field, thereby to detect the fracture in the bone.

21. The method of claim 20, wherein the bone is a first limb of a person, and said reference intensity values are intensity values previously obtained from a second limb of said person.

22. The method of claim 20, wherein the bone is a limb of a first person, and said reference intensity values are intensity values previously obtained from a limb of a second person.

23. Method for detecting a fracture in a bone, the method comprising:
obtaining reference intensity values;
generating an electric field in proximity of the bone, at a predefined frequency range, said generated electric field being substantially parallel to longitudinal axis of the bone;
measuring intensity values of said generated electric field over said predefined frequency range; and
analyzing said intensity values measured over said predefined frequency range using said obtained reference intensity values, thereby to detect fracture in the bone.

24. The method of claim 23, wherein the bone is a first limb of a person, and said reference intensity values are intensity values previously measured over a second limb of said person.

25. The method of claim 23, wherein the bone is a limb of a first person, and said reference intensity values are intensity values previously measured over a limb of a second person.
